(19) United States
(12) Patent Application Publication
(10) Pub. No.: US 2014/0274969 A1
(43) Pub. Date: Sep. 18, 2014

(54) USE OF MINOCYCLINE FOR THERAPEUTIC TREATMENT OF ACUTE SPINAL CORD INJURIES

(71) Applicant: RICK HANSEN INSTITUTE, Vancouver (CA)

(72) Inventors: Steven CASHA, Calgary (CA); John HURLBERT, Calgary (CA); Voon Wee YONG, Calgary (CA)

(73) Assignee: RICK HANSEN INSTITUTE, Vancouver (CA)

(21) Appl. No.: 13/836,380

(22) Filed: Mar. 15, 2013

(51) Int. Cl. A61K 31/65

(52) U.S. Cl. CPC A61K 31/65
USPC 514/152

(57) ABSTRACT

A dosing regime for providing therapy for a neurological trauma such as spinal cord injury in a human subject. The dosing regime comprises intravenous administration of a plurality of minocycline doses for a 7-day period at 12-h intervals such that a steady state level of minocycline is reached and maintained in a subject's serum and/or cerebrospinal fluid after administration of three minocycline doses. An initial minocycline dose of at least 800 mg is preferably administered within 12 h after an occurrence of the neurological trauma. The minocycline dosage is sequentially tapered to 400 mg and then maintained at 400 mg for the remainder of the 7-day treatment period.

### FIELD OF THE DISCLOSURE

This disclosure relates to therapies of neurological traumas such as spinal cord injuries. More particularly, this disclosure pertains to dosage regimes comprising sequential intravenous administration of minocycline doses for amelioration of neurological traumas.

### BACKGROUND

One of the most devastating medical conditions to affect present day society is that of acute spinal cord injury. This condition afflicts primarily young people, significantly degrades independence, consumes huge societal resources, and bestows life-long disability. There are currently no therapeutic interventions proven to significantly improve outcome following acute spinal cord injury (SCI).

The annual incidence of acute spinal cord injury is approximately 40-50 cases per million population per year, not including those that die immediately as a result of the injury. The prevalence of people living with spinal cord injury varies from country to country but ranges between 11-112 per 100,000. In a database of over 19,000 patients with SCI, 82% are male, the average age at injury is 31.8 years while somewhere between ½-⅔ of those afflicted are between the age of 16 and 30. The leading causes of spinal cord injury are motor vehicle accidents (45%), falls (22%), and sports (14%). Despite advances in critical care, mortality rates for those admitted with acute SCI still range from 4-17%, even when the injury is isolated to the spinal cord alone. Hospital stays of a week or longer are necessitated in approximately 10% of patients with spinal cord injuries each year whose lives are complicated by pressure sores, autonomic dysreflexia, pneumonia, atelectasis, deep venous thrombosis and renal calculi. Spasticity and pain significantly add to neurological disability in 25%. Long-term reduced life expectancy is accounted for by pneumonia, pulmonary emboli, and septicemia.

The average yearly health care and living expenses vary depending on the level and severity of the injury. In the first year after injury it is estimated that the total amount of these costs for a person with paraplegia is $194,000 (US). In contrast a person with high quadriplegia will incur over $500,000 in costs. Thereafter yearly health care and living expenses consume $10,000 to $100,000 per year, not including lost wages. A decade ago aggregate costs in the United States were estimated to range from $4-5.6 billion per year, while more recently they have been estimated at $7.7 billion.

Despite recent efforts of prevention programs such as Think First, Safe Kids, and even despite new laws mandating seat belt and air bag use, the incidence of acute spinal cord injury has not changed significantly and may actually be increasing in certain parts of the population. At present, there is no treatment proven effective at reducing or eliminating the disability resulting from acute SCI. Therapeutic strategies aimed at promoting axonal and neuronal regeneration hold the greatest promise for cure in the future, but are currently limited to modest success in relatively simple preparations spanning short distances. Improving recovery of partially injured neuronal tissue is a treatment goal with perhaps more immediate promise.

The pathophysiology of acute SCI is generally considered in two broad categories: primary and secondary injury. Primary injury can be defined as the immediate structural sequelae arising as a direct result of mechanical forces applied to the spinal cord. These forces include compression, distraction, shear and laceration. The sequelae include cell death through membrane disruption, hemorrhage, and ischemia.

Secondary injury occurs in a delayed fashion following the primary insult Evidence indicates that the cellular and subcellular events arising from primary injury set into motion secondary cascades that, over time, cause further damage to sublethally affected or possibly even undamaged neural and glial tissue. The distinction between the two processes is not precise, predominantly because even primary damage has not been well defined and likely evolves over a period of time. Controversy exists with respect to the actual contribution of secondary injury to overall outcome. Nonetheless, salvage of even a small amount of CNS tissue after SCI may have major functional repercussions; animal studies suggest only 10% of spinal cord long tract connections to be critical for locomotion.

Several potential processes of secondary injury have been studied in animal models of both cerebral ischemia and spinal cord injury. These include free radical production, lipid peroxidation, eicosanoid and prostaglandin production, neutral protease activation, intracellular ionic shifts, and excitotoxicity. More recently apoptosis, inflammation and glial activation, and intracellular protein synthesis have captured the attention of researchers. The multiple arms of investigation into these mechanisms attest to the ongoing need for an effective therapeutic strategy to treat acute SCI. Until regeneration strategies are perfected, limitation of secondary cell death is the only avenue of hope for patients affected with this devastating condition.

### SUMMARY OF THE DISCLOSURE

The exemplary embodiments of the present disclosure pertain to dosage regimes for delivery of therapeutic treatments for ameliorating the physiological and motor activity debilitations resulting from severe neurological traumas such as those exemplified by spinal cord injuries. Some embodiments pertain to dosage regimes comprising intravenous administration of minocycline at 12-h intervals for a 7-day period whereby target steady state minocycline levels are established after administration of three doses to a human subject. The exemplary dosage regimes are preferably started as soon as clinically possible after the occurrence of a severe neurological trauma, for example within 12 hours or sooner, within 24 hours or sooner, within 36 hours or sooner. The initial minocycline dose is at least 800 mg and is tapered by 100 mg in successive dosage until the 400-mg dose, after which, the remaining doses in the dosage regimes comprise 400 mg minocycline. Some exemplary embodiments of this disclosure pertain to methods for therapeutic treatment of severe neurological traumas using the minocycline dosage regimes disclosed herein.

### DESCRIPTION OF THE DRAWINGS

The present disclosure will be described in conjunction with reference to the following drawings in which:

FIG. 1 is a schematic flowchart of subject enrolment, randomization, allocation to treatment groups, and follow up;

FIG. 2(A) is a chart showing the pharmacokinetics of minocycline in serum, and FIG. 2(B) is a chart showing the pharmacokinetics of minocycline in cerebrospinal fluid;

FIG. 3 is a chart showing motor recovery, using ASIA scores, after complete spinal cord injury, incomplete spinal cord injury, and central cord spinal cord injury;

FIGS. 4(A)-4(E) are charts showing the effects of minocycline on changes in ASIA motor scores in (A) all subjects, (B) thoracic injured subjects, (C) cervical injured subjects, (D) subjects with cervical motor-complete injury, and (E) subjects with cervical motor-incomplete injury;

FIGS. 5(A)-5(C) are charts showing the effects of minocycline dosage rates on motor recovery in (A) cervical injured subjects, (B) subjects with cervical motor-complete injury, and (C) subjects with cervical motor-incomplete injury;

FIGS. 6(A)-6(D) are charts showing the effects of minocycline on (A) upper extremity motor recovery after cervical spinal cord injury, (B) lower extremity motor recovery after cervical spinal cord injury, (C) motor recovery in zones of partial preservation after cervical complete injury, and (D) motor recovery in new segments after cervical complete injury;

FIGS. 7(A)-7(C) are charts showing the effects of minocycline on ASIA light touch sensory scores in (A) subjects with cervical complete spinal cord injury, (B) subjects with cervical incomplete spinal cord injury, and (C) subjects with thoracic spinal cord injury;

FIGS. 8(A)-8(C) are charts showing the effects of minocycline on ASIA pinprick sensory scores in (A) subjects with cervical complete spinal cord injury, (B) subjects with cervical incomplete spinal cord injury, and (C) subjects with thoracic spinal cord injury;

FIGS. 9(A)-9(C) are charts showing the effects of minocycline on changes in functional outcomes assessed with the Spinal Cord Independence Measure (SCIM) in (A) all subjects, (B) subjects with cervical complete spinal cord injury, and (C) subjects with cervical incomplete spinal cord injury;

FIGS. 10(A)-10(B) are charts showing the effects of minocycline on changes in functional outcomes assessed with the Functional Independence Measure Motor score (FIMM) in (A) all subjects, (B) subjects with cervical complete spinal cord injury, and (C) subjects with cervical incomplete spinal cord injury;

FIGS. 11(A)-11(B) are charts showing the effects of minocycline on changes in functional outcomes assessed with the London Handicap Score (LHS) in (A) all subjects, (B) subjects with cervical complete spinal cord injury, and (C) subjects with cervical incomplete spinal cord injury; and

FIGS. 12(A)-12(B) are charts showing the effects of minocycline on changes in functional outcomes assessed with the Short Form 36 (SF36) physical score in (A) all subjects, (B) subjects with cervical complete spinal cord injury, and (C) subjects with cervical incomplete spinal cord injury.

### DETAILED DESCRIPTION OF THE DISCLOSURE

Unless defined otherwise, all technical and scientific terms used herein have the meanings that would be commonly understood by one of skill in the art in the context of the present specification. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

As used herein and in the appended claims, the singular forms “a,” “an,” and “the” include plural references unless the context clearly dictates otherwise. Thus, for example, reference to “the agent” includes reference to one or more agents and equivalents thereof known to those skilled in the art, and so forth.

The terms “optional” or “optionally” or “alternatively” as used herein, mean that the subsequently described event, circumstance, or material may or may not occur or be present, and that the description includes instances where the event, circumstance, or material occurs or is present and instances where it does not occur or is not present.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also, encompassed within the disclosure, subject to any specifically excluded limit in the stated range. Where the stated range includes one or both of the limits, ranges excluding either or both of those included limits are also included in the disclosure.

The terms “inhibit”, “inhibiting”, and “inhibition” as used herein, mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between the specifically recited percentages, as compared to native or control levels.

The terms “promote”, “promotion”, and “promoting” as used herein, refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, the increase in an activity, response, condition, disease, or other biological parameter can be a 10, 20, 30, 40, 50, 60, 70, 80, 90, 100%, or more, including any amount of increase in between the specifically recited percentages, as compared to native or control levels.

The term “ameliorate” as used herein, means to diminish the negative effects of an injury or a trauma, or alternatively means to make better or to improve a physiological condition that has been diminished by an injury or a trauma, or alternatively means to make better or to improve a motor function that has been diminished by an injury or a trauma.

As used herein, the term “subject” means any target of administration. The subject can be a vertebrate, for example, a mammal. Thus, the subject can be a human. The term does not denote a particular age or sex. Thus, adult, juvenile, and newborn subjects, whether male or female, are intended to be covered. A patient refers to a subject afflicted with a disease or disorder. The term “patient” includes human and veterinary subjects.

The term “stratum” as used herein, means a class or group to which a subject has been assigned.

The term “spinal cord injury” as used herein means to any injury to the spinal cord that is caused by trauma instead of disease. Depending on where the spinal cord and nerve roots are damaged, the symptoms can vary widely, for example from pain to paralysis to incontinence. Spinal cord injuries are described at various levels of “incomplete”, which can vary from having no effect on the patient to a “complete” injury which means a total loss of function. Spinal cord injuries have many causes, but are typically associated with major trauma from motor vehicle accidents, falls, sports injuries, and violence. The abbreviation “SCI” means spinal cord injury.

The abbreviation “ASIA” means the American Spinal Injury Association.

The term “ASIA score” means the is the score developed by the American Spinal Injury Association for the essential minimal elements of neurologic assessment for all patients with a spinal injury. These minimal elements are strength assessment of ten muscles on each side of the body and pin-prick discrimination assessment at 28 specific sensory locations on each side. An ASIA score “A” means complete impairment evidenced by no motor or sensory function in the lowest sacral element (S4-S5). An ASIA score “B” means incomplete impairment evidenced by sensory function below the neurologic level and in sacral element S4-S5, and no motor function below the neurologic level. An ASIA score “C” means incomplete impairment evidenced by motor function preserved below the neurologic level and more than half of the key muscle groups below the neurologic level have a muscle grade of less than 3. An ASIA score “D” means incomplete impairment evidenced by motor function preserved below the neurologic level and at least half of the key muscle groups below the neurologic level have a muscle grade greater than 3. An ASIA score “E” means sensory and motor function is normal.

The term “central cord syndrome” as used herein refers to an acute cervical spinal cord injury that accounts for approximately 9% of traumatic spinal cord injury. Central cord syndrome is characterized by disproportionately greater motor impairment in upper compared to lower extremities, and variable degree of sensory loss below the level of injury in combination with bladder dysfunction and urinary retention. This syndrome differs from that of a complete lesion, which is characterized by total loss of all sensation and movement below the level of the injury. Consequently, central cord syndrome is generally associated with favorable prognosis for some degree of neurological and functional recovery. The abbreviation “CCS” means central cord syndrome.

The term “central nervous system” as used herein means the brain, spinal cord, and a complex network of neurons interconnected with and communicating with the peripheral nervous system. The abbreviation “CNS” means central nervous system.

The term “cerebrospinal fluid” as used herein refers to a clear colorless bodily fluid produced in the choroid plexus of the brain. Cerebrospinal fluid acts as a cushion or buffer for the cortex, providing a basic mechanical and immunological protection to the brain inside the skull and serves a vital function in cerebral autoregulation of cerebral blood flow. The cerebrospinal fluid occupies the subarachnoid space and the ventricular system around and inside the brain and spinal cord. Cerebrospinal fluid constitutes the content of the ventricles, cisterns, and sulci of the brain, as well as the central canal of the spinal cord. The abbreviation “CSF” means cerebrospinal fluid.

As used herein, the terms “treatment,” “treating” and the like, refer to obtaining a desired pharmacologic and/or physiologic effect. The effect may be therapeutic in terms of a partial or complete cure for an injury and/or adverse affect attributable to the injury.

The term “treatment” as used herein, covers any treatment of SCI in a mammal, particularly in a human, and includes: (a) inhibiting SCI, i.e., arresting its development; and (c) relieving SCI, i.e., causing regression of the injury.

The term “matrix metalloproteinases” as used herein means a family of proteases capable of degrading extracellular matrix proteins, and additionally, are known to be involved in the cleavage of cell surface receptors, the release of apoptic ligands e.g. the FAS ligand, and the activation and inactivation of chemokines and cytokines. The abbreviation “MMP” means matrix metalloproteinases.

The family of matrix metalloproteinases (MMPs) consists of over 23 members, designated according to numerals e.g. MMP-1, MMP-2, etc., or alternatively, by categories e.g. the gelatinase subgroup of MMP-2, the gelatinase group of MMP-9 etc. Together, MMP family members can degrade all protein components of the extracellular matrix (ECM). They are thus important in processes that involve matrix remodeling such as developmental tissue morphogenesis or wound healing. MMPs also regulate growth factor bioavailability, to possess anti- and pro-inflammatory properties, are involved in cellular signal transduction, and play key roles in mediating cell survival and death.

The activity of most MMP members is normally negligible or low in the adult CNS, but a substantial upregulation of MMPs has been documented in several neurological disorders including multiple sclerosis, stroke and malignant gliomas. These upregulated MMPs are thought to contribute to CNS pathology by virtue of disrupting the blood-brain barrier, destroying CNS myelin, promoting CNS inflammation, and by acting as neurotoxins.

Evidence is beginning to accumulate linking MMPs to CNS injury. Several MMPs have been found to be upregulated after CNS trauma in a mouse cortical resection injury model. The elevation has been attributed to transcriptional induction by an inflammatory cytokine that is expressed soon after injury, interleukin-1 (IL-1). In a cortical impact model MMP-9 genetically deficient mice had fewer motor deficits than wild-type controls. In addition, Nissl-stained histological sections demonstrated lesion volume to be smaller in the MMP-9 null mice compared to controls.

Similar experiments implicate MMPs in the pathology of SCI. De Castro et al. (2000, Metalloproteinase increases in the injured rat spinal cord. Neuroreport 11:3551-3554) reported an increase of MMP-2 and -9 protein levels after injury to the spinal cord of rats. MMP-9 levels were maximal at 12-24 h, while MMP-2 peaked 5 days post-injury. Infiltrating neutrophils were found to be the principal source of MMP-9 although CNS sources were not ruled out. Xu et al. (2001, Glucocorticoid receptor-mediated suppression of activator protein-1 activation and matrix metalloproteinase expression after spinal cord injury. J. Neurosci. 21:92-97) found increased expression of MMP-1 and -9 transcripts in SCI. This expression was inhibited by the treatment of animals with methylprednisolone. Using a process called in-situ zymography, in which the net proteolytic activity was measured in a mixture of expression of MMPs and their endogenous inhibitors, gelatinase activity was found to be increased in the spinal cord after a contusion injury in rats (Duchossoy et al., 2001, Matrix metalloproteinases: potential therapeutic target in spinal cord injury. Clin. Chem. Lab. Med. 39:362-367). Prominent and sustained elevation of MMP-12 has been observed in the spinal cord of mice following a compression injury. Furthermore, SCI in mice genetically deficient for MMP-12 resulted in a more favorable behavioral outcome (hind limb movement) than a similar injury to wild-type controls (Wells et al., 2003, An adverse role for matrix metalloproteinase (MMP)-12 following spinal cord injury in mice. J. Neurosci. 23:10107-10115).

Minocycline is a broad-spectrum second-generation tetracycline antibiotic. It is a bacteriostatic antibiotic, classified as a long-acting type. Orally administered compositions of minocycline are commonly used for treatment of acne vulgaris. Oral compositions of minocycline are also used clinically for treatment skin diseases caused by methicillin-resistant Staphylococcus aureus, Lyme disease, asthma, and rheumatoid arthritis. Commonly prescribed dosages for oral administration of minocycline are in the range of about 50 to 300 mg/day, about 75 to 225 mg/day, about 100 to 150 mg/day.

Minocycline is available as a HCL salt or hydrate. The molecular formula for minocycline is C₂₃H₂₇N₃O₇.HCl with a molecular weight of 493.95. Pirenzepine's IUPAC name is (2E,4S,4aR,5aS,12aR)-2-(amino-hydroxy-methylidene)-4,7-bis(dimethylamino)-10,11,12a-trihydroxy-4-a,5,5a,6-tetrahydro-4H-tetracene-1,3,12-trione.

Minocycline has been in commercial use for human administration for over 30 years. Clinical pharmacokinetics and safety have been well described and are detailed in a comprehensive summary (Saivin et al., 1988, Clinical Pharmacokinetics of Doxycycline and Minocycline. Clinical Pharmacokinetics 15: 355-366). It has the highest partition coefficient of the tetracyclines and thus is highly lipophillic. This allows for relatively easy penetration into the central nervous system, independent of inflammatory disease. Peak serum concentrations of minocycline are observed about 2 to 3 hours after oral administration, for example about 2 mg/L after administration of 150 mg of minocycline and about 4 mg/L after administration of 300 mg of minocycline. Intravenous administration of 200 mg results in peak concentrations of minocycline in serum of about 4 mg/L to about 6 mg/L one to two hours after administration.

Elimination of minocycline in its biologically active form occurs primarily through the feces and represents 20-35% of the initial dose. Eight to twelve percent is eliminated unchanged through the kidneys. Three microbiologically inactive metabolites are produced in the liver and are also excreted in the urine and feces. This route of metabolism and excretion accounts for approximately 50-60% of the administered dose. The half-life of minocycline is 16 hours following oral or intravenous administration. These pharmacokinetics are not appreciably altered by renal failure, nor does minocycline harbor the catabolic characteristics of first generation tetracyclines. Similarly neither forced diuresis, peritoneal dialysis, nor hemodialysis modifies pharmacokinetics. Liver cirrhosis does not require modification of dosages. Hence minocycline administration is not contraindicated in either renal or liver failure.

Side effects have been associated with minocycline administration. The vast majority are non-specific and transitory. Resolution occurs quickly with cessation of the drug. The symptoms can be classified into two main categories: vestibular dysfunction and gastrointestinal upset. Vestibular dysfunction manifests itself as light-headedness, dizziness, vertigo, and rarely fainting spells. Gastrointestinal upset includes anorexia, nausea, vomiting, diarrhea, and constipation. In addition stomatitis, glossitis, dysphagia, and pruritis ani have also been reported. Side effects specific to intravenous as opposed to oral administration have not been encountered.

More serious adverse events that have been associated with long-term minocycline administration include hypersensitivity syndrome reaction, serum sickness-like reaction, drug induced lupus, and single organ dysfunction such as pneumonitis, cutaneous eruption, and hepatitis. These reactions typically occur weeks to years after institution of treatment. The overall incidence is extremely low. Despite millions of prescriptions for minocycline globally each year, only 19 cases of hypersensitivity reaction, 11 cases of serum sickness-like reaction, 33 cases of drug induced lupus, and 40 cases of single organ dysfunction could be found. Although a true denominator is not known, the incidence of these serious adverse reactions is estimated to be somewhere between 1 in 10,000 to 1 in 1,000,000. Treatment for these reactions has not been standardized and is generally supportive in nature.

In summary, minocycline is absorbed readily following oral administration and can be safely given intravenously. Because of its lipophilicity, minocycline penetrates well into the CNS. In reference to published scientific research reports, it is known that serum minocycline concentrations of up to 6.2 mg/L are well tolerated in humans. Early side effects consist primarily of vestibular and gastrointestinal symptoms and respond well to drug withdrawal. More serious side effects such as hypersensitivity, drug induced lupus, and single organ failure are rare, and typically associated with long-term administration.

It is also known that minocycline is a potent inhibitor of MMP activity (Golub et al., 1984, Tetracyclines inhibit tissue collagenase activity: A new mechanism in the treatment of periodontal disease. J. Periodontal Res. 19:651-655; Paemen et al., 1996, The gelatinase inhibitory activity of tetracyclines and chemically modified tetracycline analogues as measured by a novel microtiter assay for inhibitors. Biochem. Pharma. 52:105-111) and also, to attenuates MMP production.

Glutamate excitoxicity has been implicated as a major mediator of cell death in the CNS. Some studies have shown that minocycline may reduce excitotoxicity in mixed neuronal and glial spinal cord cultures treated with glutamate, kainate, and NMDA (Tikka et al. 2001, Minocycline, a tetracycline derivative, is neuroprotective against excitotoxicity by inhibiting activation and proliferation of microglia. J. of Neurosci. 21(8):2580-2588; Tikka and Koistinaho, 2001, Minocycline provides neuroprotection against N-Methyl-D-aspartate neurotoxicity by inhibiting microglia. J. Immunol. 166:7527-7533). Application of 20 nM Minocycline 30 minutes before 24-hour excitotoxin exposure more than doubled neuronal cell survival approaching uninjured control counts. Inhibition of microglia proliferation was noted along with reduced IL-1β and NO metabolite production.

Some clinical studies with minocycline in human neurological disease have shown encouraging results. For example, Plane et al. (2010, Prospects for minocycline neuroprotection. Arch. Neurol. 67:1442-1448). However, other studies have shown that minocycline was associated with clinical deterioration (Gordon et al., 2007, Placebo-controlled phase I/II studies of minocycline in amyotrophic lateral sclerosis. Neurology 62:1845-1847).

The potential benefits of minocycline for therapeutic treatment of traumatic SCI have been assessed using animal models. Some studies have shown that post-trauma minocycline could facilitate significant recovery from SCI in mice and rats. For example, Wells et al., found that two 50 mg/kg intraperitoneal doses of minocycline administered at 24 h intervals immediately after SCI followed by three 25 mg/kg doses at 24 h intervals facilitated significant recovery from SCI in mice (2003, Neuroprotection by minocycline facilitates significant recovery from spinal cord injury in mice. Brain 126:1628-1637). Lee et al., found that one 90 mg/kg dose of minocycline immediately after SCI followed by 45 mg/kg doses every 12 h improved functional motor control in the hind limbs of rats and reduced the size of lesions formed on their spinal cords (2003, Minocycline reduces cell death and improves functional recovery after traumatic spinal cord injury in the rat. J. Neurotrauma 20:1017-1027). Festoff et al. (2006, Minocycline neuroprotects, reduces microgliosis, and inhibits caspase protease expression early after spinal cord injury. J. Neurochem. 97:1314-1326) found that three sequential 30 mg/kg intraperitoneal doses of minocycline administered: (i) 30 min after SCI, (ii) 60 min after SCI, and (iii) 24 h after SCI, facilitated significant recovery in functional hind limb motor control in rats over a 28-day post SCI observation period. Festoff et al. also observed that a single 90 mg/kg intraperitoneal dose administered at one of (i) 30 min after SCI, (ii) 60 min after SCI, and (iii) 24 h after SCI, provided similar effects to the three sequential doses. Yune et al. (2007, Minocycline alleviates death of oligodendrocytes by inhibiting pro-nerve growth factor production in microglia after spinal cord injury. J. Neurosci. 27:7751-7761) found that sequential intraperitoneal injections of minocycline at 12-h intervals after SCI for a 3-day period beginning with an initial 90 mg/kg dose followed by 30 mg/kg doses, significantly increased hindlimb motor function in rats.

On the other hand, numerous studies have shown that administration of minocycline in multiple doses has significant negative effects in various animal models. For example, Yang et al. (2003. Minocycline enhances MPTP toxicity to dopaminergic neurons. J. Neurosci. Res. 74:278-285) demonstrated that intraperitoneal administration of (a) two 45 mg/kg doses of minocycline followed by two 22.5 mg/kg doses at 12-hour intervals, and (b) one 60 mg/kg dose of minocycline followed by two 30 mg/kg doses at 12-hour intervals, significantly exacerbated the toxic effects of MPTP (1-methyl-4-phenyl-1,2,3,6-tetrahydropyridine) on neural function in mice. Tsuji et al. (2004, Minocycline worsens hypoxic-ischemic brain injury in a neonatal mouse model. Exp. Neurol. 189:58-65) showed that subcutaneous administration of a 135-mg/kg dose of minocycline followed by two 68 mg/kg doses at 24-h intervals significantly exacerbated the effects of hypoxic-ischemic brain in mice. Pinzon et al. (2008, A re-assesment of minocycline as a neuroprotective agent in a rat spinal cord contusion model. Brain Res. 1243:146-151) assessed two routes of minocycline dosing on restoration of functional motor activity in rats that received contusive SCI. The first route was intraperitoneal administration of 90 mg/kg immediately after SCI followed by two intraperitoneal doses of 45 mg/kg at 12-h intervals. The second route was administration of a first dose of 90 mg/kg via jugular annula immediately after SCI followed by two intraperitoneal doses of 45 mg/kg at 12-h intervals. Pinzon et al. reported that neither route for minocycline administration resulted in any behavioural or histological improvements or recovery from SCI in rats. Lee et al. (2010, Lack of neuroprotective effects of simvastatin and minocycline in a model of cervical spinal cord injury. Exp. Neurol. 225:219-230) found that intraperitoneal injections of 90 mg/kg of minocycline at daily intervals for 3 days after cervical SCI in a rat model, did not result in any functional or histological improvements over a 42-day post-SCI monitoring.

In view of these and other conflicting and contradictory medical and scientific reports known to those skilled in this art regarding the potential benefits of minocycline for ameliorating the effects of SCI and other forms of neurological trauma, we have surprisingly discovered that a 7-day twice-daily minocycline dosing regimen comprising an initial highly elevated dose administered intravenously within 12 h of an occurrence of a traumatic neurological injury followed by tapering of the subsequent minocycline doses to a target base level over the initial 2½ day dosing period, provides significant post-trauma recovery and at least partial restoration of motor function in human subjects. Accordingly, one embodiment of the present disclosure pertains to an exemplary dosing regimen comprising an initial minocycline dose of about 800 mg administered within about 12 h of the occurrence of a neurological trauma incident, a second minocycline dose of about 700 mg administered about 12 h after administration of the initial first dose, a third minocycline dose of about 600 mg administered about 12 h after administration of the second dose, a fourth minocycline dose of about 500 mg administered about 12 h after administration of the third dose, a fifth minocycline dose of about 400 mg administered about 12 h after administration of the first dose, and then administering 400 mg minocycline dosages at about 12-h intervals for the duration of the 7-day dosing regimen. It is preferable, if clinically possible, to commence administration of the 7-day minocycline dosing regimen as soon possible after the occurrence of the neurological trauma. However, if it is not possible to commence the dosing regimen within about 12 h of the occurrence of the neurological trauma, the dosing regimen may commence as soon as it is clinically feasible and possible, for example within 16 h, 18 h, 20 h, 22 h, 24 h, 28 h, 32 h, 36 h after the occurrence of the neurological trauma. Samples of serum and CSF can optionally be collected from a subject receiving minocycline doses according to the exemplary dosing regimes disclosed herein at multiple sampling periods during the 7-day regimen period, and assayed for the presence of minocycline to confirm achievement of steady state levels. A suitable minocycline steady state level in a subject's serum after administration of three doses is in a range of about 7 μg/ml to about 17 μg/ml. A suitable minocycline steady state level in a subject's CSF after administration of three doses is in a range of about 1.5 μg/ml to about 3.5 μg/ml. The dosage concentrations subsequent to the initial dose can be adjusted during the dosage regime as necessary to maintain minocycline steady state levels in a subject's serum and their CSF.

It should be noted that the extent and degree of post-trauma recovery and restoration of motor function after a traumatic neurological injury, for example SCI, from use of the exemplary dosage regimes disclosed herein may be diminished with increasing delays in administration of the initial minocycline dose. However, it may still be possible to ameliorate a potential diminishing in the restorative effects of minocycline resulting in delayed commencement of the dosing regime beyond the target 12-h post-trauma occurrence window, by increasing the initial dose of minocycline administered. In such circumstances, for example, an initial dose administered within about 20 hrs after the occurrence of a neurological trauma, may comprise about 1,000 mg or about 900 mg minocycline and then sequentially tapered at 12-h intervals to about 400 mg with the fifth dose. It should be noted that the selected initial and subsequent minocycline doses comprising the 7-day dosing regime should enable establishment and maintenance of minocycline steady state levels after administration of the third dose, with a target steady state in the subject's serum of about 7 μg/ml to about 17.5 μg/ml, and in their CSF of about 1.5 μg/ml to about 3.0 μg/ml.

The term “therapeutically effective” as used herein, means that the amount of a minocycline composition used is of sufficient quantity to ameliorate one or more effects or symptoms of SCI. Such amelioration only requires a reduction or alteration, not necessarily elimination. The “therapeutically effective amount” will vary depending on the compound, the disease and its severity and the age, weight, etc., of the subject to be treated.

As used herein, “pharmaceutical composition” includes any composition for parenteral administration of minocycline to a subject in need of therapy for SCI. Pharmaceutical compositions may include carriers, diluents, buffers, preservatives, surface active agents and the like in addition to minocycline. Pharmaceutical compositions may also include one or more active ingredients such as antimicrobial agents, anti-inflammatory agents, anaesthetics, and the like.

The term “unit dosage form” as used herein, refers to physically discrete units suitable as unitary dosages for human and animal subjects, each unit containing a predetermined quantity of minocycline calculated in an amount sufficient to produce the desired effect in association with a pharmaceutically acceptable diluent, carrier or vehicle.

The term “carrier” means a compound, composition, substance, or structure that, when in combination with minocycline, aids or facilitates preparation, storage, administration, delivery, effectiveness, selectivity, or any other feature of minocycline for its intended use or purpose. For example, a carrier can be selected to minimize any degradation of minocycline and to minimize any adverse side effects in the subject.

Suitable pharmaceutically acceptable carriers include essentially chemically inert and nontoxic pharmaceutical compositions that do not interfere with the effectiveness of the biological activity of the pharmaceutical composition. Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of suitable pharmaceutical carriers include, but are not limited to, saline solutions, glycerol solutions, ethanol, N-(1(2,3-dioleyloxy)propyl)-N,N,N-trimethylammonium chloride (DOTMA), diolesylphosphotidylethanolamine (DOPE), and liposomes. Such pharmaceutical compositions should contain a therapeutically effective amount of the compound, together with a suitable amount of carrier so as to provide the form for proper administration to the subject. The formulation should suit the mode of administration.

The term “excipient” herein means any substance, not itself a therapeutic agent, which may be used in a composition for delivery of minocycline to a subject or alternatively combined with minocycline (e.g., to create a pharmaceutical composition) to improve its handling or storage properties or to permit or facilitate formation of a dose unit of the composition. Excipients include, by way of illustration and not limitation, binders, solvents, penetration enhancers, solubilizing agents, wetting agents, antioxidants, lubricants. Any such excipients can be used in any dosage forms according to the present disclosure. The foregoing classes of excipients are not meant to be exhaustive but merely illustrative as a person of ordinary skill in the art would recognize that additional types and combinations of excipients could be used to achieve the desired goals for delivery of minocycline.

In one embodiment, the pharmaceutical compositions disclosed herein comprise minocycline in a total amount by weight of the composition of about 0.1% to about 95%. For example, the amount of minocycline by weight of the pharmaceutical composition may be about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 85%, about 90% or about 95%.

Another embodiment pertains to pharmaceutical compositions comprising minocycline formulated for parenteral administration by injection. The injectable pharmaceutical compositions of the present disclosure comprise a suitable carrier solution exemplified by sterile water, saline, and buffered solutions at physiological pH. Suitable buffered solutions are exemplified by Ringer's dextrose solution and Ringer's lactated solutions. The carrier solution may comprise in a total amount by weight of about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, about 6.0%, about 6.1%, about 6.2%, about 6.3%, about 6.4%, about 6.5%, about 6.6%, about 6.7%, about 6.8%, about 6.9%, about 7.0%, about 7.1%, about 7.2%, about 7.3%, about 7.4%, about 7.5%, about 7.6%, about 7.7%, about 7.8%, about 7.9%, about 8.0%, about 8.1%, about 8.2%, about 8.3%, about 8.4%, about 8.5%, about 8.6%, about 8.7%, about 8.8%, about 8.9%, about 9.0%, about 9.1%, about 9.2%, about 9.3%, about 9.4%, about 9.5%, about 9.6%, about 9.7%, about 9.8%, about 9.9% or about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, or about 95%.

According to another aspect, the injectable pharmaceutical compositions may additionally incorporate one or more of anti-oxidants, chelating agents and the like.

The injectable pharmaceutical compositions may be presented in unit-dose or multi-dose containers exemplified by sealed ampules and vials. The injectable pharmaceutical compositions may be stored in a freeze-dried (lyophilized) condition requiring the addition of a sterile liquid carrier, e.g., sterile saline solution for injections, immediately prior to use.

The pharmaceutical compositions described herein are used in a “pharmacologically effective amount.” A “pharmacologically effective amount” is the amount of minocycline in the composition which is sufficient to deliver a therapeutic amount of the active agent during the dosing interval in which the pharmaceutical composition is administered. Accordingly, the amount of the pharmaceutical composition administered to deliver a therapeutically effective amount of minocycline is about 0.01 g, about 0.05 g, about 0.1 g, about 0.2 g, about 0.3 g, about 0.4 g, about 0.5 g, about 0.6 g, about 0.7 g, about 0.8 g, about 0.9 g, about 1 g, about 1.1 g, about 1.2 g, about 1.3 g, about 1.4 g, about 1.5 g, about 1.6 g, about 1.7 g, about 1.8 g, about 1.9 g, about 2 g, about 2.1 g, about 2.2 g, about 2.3 g, about 2.4 g, about 2.5 g, about 2.6 g, about 2.7 g, about 2.8 g, about 2.9 g, about 3 g, about 3.1 g, about 3.2 g, about 3.3 g, about 3.4 g, about 3.5 g, about 3.6 g, about 3.7 g, about 3.8 g, about 3.9 g, about 4 g, about 4.1 g, about 4.2 g, about 4.3 g, about 4.4 g, about 4.5 g, about 4.6 g, about 4.7 g, about 4.8 g, about 4.9 g, about 5 g, about 5.1 g, about 5.2 g, about 5.3 g, about 5.4 g, about 5.5 g, about 5.6 g, about 5.7 g, about 5.8 g, about 5.9 g, about 6 g, about 6.1 g, about 6.2 g, about 6.3 g, about 6.4 g, about 6.5 g, about 6.6 g, about 6.7 g, about 6.8 g, about 6.9 g, about 7 g, about 7.1 g, about 7.2 g, about 7.3 g, about 7.4 g, about 7.5 g, about 7.6 g, about 7.7 g, about 7.8 g, about 7.9 g, about 8 g, about 8.1 g, about 8.2 g, about 8.3 g, about 8.4 g, about 8.5 g, about 8.6 g, about 8.7 g, about 8.8 g, about 8.9 g, about 9 g, about 9.1 g, about 9.2 g, about 9.3 g, about 9.4 g, about 9.5 g, about 9.6 g, about 9.7 g, about 9.8 g, about 9.9 g or about 10 g.

As noted previously, it is desirable to establish after administration of the third minocycline dose, a steady-state level of minocycline in a subject's serum of about 7 μg/ml to about 17.5 μg/ml, and a steady-state level of minocycline in their CSF of about 1.5 μg/ml to about 3.0 μg/ml. Accordingly, while it is preferable to administer the dosing regimes according to the dosage levels and time intervals disclosed herein via intravenous injections, the dosage regimes may be administered using other parenteral dosing routes as exemplified by intrathecal injections, intraosseous injections, intraperitoneal injections, intramuscular injections, among others. It is also optional if so desired or so necessitated, to administer the dosing regimes orally. Depending on a subject's situation and condition, a dosing regime according to the present disclosure may be administered with a plurality of delivery routes.

For example, the dosing regime may be administered using intravenous injections for a first group of doses, and then with one or more alternative dosing routes for one or more groups of doses making up the remainder of 7-day twice daily dosage regime. The most important consideration is to achieve a steady-state of minocycline in the subject's serum and/or CSF within a target range as quickly as possible and then to maintain the minocycline steady state levels in the subject's CSF and serum for the duration of the 7-day dosing regime.

The following example is provided to more fully describe the disclosure and are presented for non-limiting illustrative purposes.

### EXAMPLE

### Materials and Methods

The research protocol disclosed herein was approved by the University of Calgary Conjoint Health Research Ethics Board. Between June 2004 and August 2008, all subjects presenting with motor deficit secondary to acute traumatic SCI to the Spine Service at the Foothills Medical Centre in Calgary, Calgary, Alberta, Canada, were immediately identified to the principal investigators and were assessed and screened for this trial (Clinical Trials Gov. Identifier No. NCT00559494). Those within 12 h of injury who met other inclusion criteria (Table 1) were offered enrolment.

TABLE 1
Inclusion and exclusion criteria

| Inclusion criteria | Exclusion criteria |
|---|---|
| Age 16 or over; | Tetracycline hypersensitivity; |
| SCI with ASIA level between C0 and | Elevated liver function tests (AST, ALT, |
| T11, and resulting in a detectable change | alkaline phosphatase, or total bilirubin |
| in the ASIA motor assessment; | greater than 2 times the upper limit of |
| English speaking subject able to provide | normal); |
| informed consent; | History of systemic lupus erythematosus |
| Randomization and administration of | (SLE); |
| first dose (drug or placebo) within 12 h | Pre-existing hepatic or renal disease; |
| of injury. | Pregnancy or breast feeding; |
| | Presence of systemic disease that might |
| | interfere with patient safety, compliance |
| | or evaluation of the condition under |
| | study (e.g. insulin-dependent diabetes, |
| | Lyme disease, clinically significant |
| | cardiac disease, HIV, HTLV-1); |
| | Associated traumatic conditions |
| | interfering with informed consent or |
| | outcome assessment (e.g. closed head injury). |

TABLE 2

| | Minocycline | Placebo |
|---|---|---|
| Variable | (n = 27) | (n = 25) |
| Mean age (years) | 40.9 | 32.1 |
| Sex (male/female) | 22/5 | 17/8 |
| Mechanism of injury | | |
| Motor vehicle collision | 14 | 16 |
| Work place accident | 5 | 4 |
| Sport injury | 6 | 3 |
| Fall | 2 | 2 |
| Severity and level of injury | | |
| A: Cervical | | |
| (i) Motor-complete | 7 | 13 |
| Motor level C1-4 | | |
| ASIA A | 6 | 6 |
| ASIA B | | 2 |
| Motor level C5-8 | | |
| ASIA A | 1 | 2 |
| ASIA B | | |
| (ii) Motor-incomplete | 5 | 4 |
| Motor level C1-4 | | |
| ASIA C | 1 | 1 |
| ASIA D | 1 | 1 |
| Motor level C5-8 | | |
| ASIA C | 3 | 1 |
| ASIA D | | 1 |
| (iii) Central cord injury | 4 | 2 |
| Motor level C1-4 | | |
| ASIA C | 1 | |
| ASIA D | 1 | 1 |
| Motor level C5-8 | | |
| ASIA C | 2 | |
| ASIA D | 1 | |
| B: Thoracic | | |
| (iv) Motor-complete | 11 | 5 |
| Sensory level T1-6 | | |
| ASIA A | 6 | 3 |
| Sensory level T7-12 | | |
| ASIA A | 5 | 2 |
| (v) Motor-incomplete | 0 | 1 |
| Sensory level T7-12 | | |
| ASIA C | | 1 |
| Post-injury time of first dose (h) | 10.7 | 9.6 |
| Time to spinal decompression (h) | 16.5 | 18.3 |
| Spinal cord perfusion pressure (SCPP) | | |
| SCPP | 11 | 10 |
| Control | 11 | 12 |
| Not randomized* | 5 | 3 |

*Patients who did not exhibit spinal cord perfusion pressure less than 75 mmHg were not randomized.

Following written informed consent and prior to randomization, 52 patients were stratified into three groups that were predicted to behave differently within the study: (i) 36 patients presenting motor-complete SCI (ASIA A or B); (ii) 10 patients presenting motor-incomplete SCI (ASIA C or D); and (iii) 6 patients presenting central cord syndrome (ASIA C or D with mean lower extremity motor scores 4 upper extremity). The baseline characteristics for the patients are shown in Table 2.

All subjects received an indwelling lumbar catheter (at L4/5) for CSF sampling and CSF pressure monitoring, a radial arterial line for blood pressure monitoring and a subclavian central venous catheter. Augmentation of spinal cord perfusion, anticipated to be a confounding variable, was controlled through a second randomization as detailed below. Surgical decompression and stabilization was performed within 24 h of injury. Subjects were not treated with corticosteroids. All subjects were screened and enrolled in the Foothills Medical Centre emergency department and were subsequently managed in the intensive care and neuro-surgery in-patient ward. Then, they were transferred to the University of Calgary Spinal Cord Injury Rehabilitation Programme, also housed at the Foothills Medical Centre.

### Randomization and Masking

Subjects were randomized (1:1) to receive intravenous minocycline doses (Wyeth Pharmaceuticals) or placebo doses (equal volumes of normal saline) in blocks of 10. For this purpose, sets of 10 random numbers balanced for odd and even integers were computer generated. Sequentially numbered and sealed packaged kits containing minocycline or placebo were constructed from the randomization codes for each stratified group by an independent individual not otherwise involved in the trial. Patients were administered the next available treatment kit for their appropriate treatment group. With the exception of the bedside nurse responsible for study drug administration, all subjects and research personnel were blinded to treatment until the end of the study (FIG. 1).

### Procedures

The first five subjects randomized to the minocycline group were administered 200 mg doses twice-daily, the maximum previously reported human minocycline dose (Macdonald et al., 1973, Pharmacokinetic studies on minocycline in man. Clin. Pharmacol. Ther. 14:852-61; Carney et al., 1974, Minocycline excretion and distribution in relation to renal function in man. Clin. Exp. Pharmacol. Physiol. 1:299-308). Serum and CSF minocycline levels were assayed (FIGS. 2(A), 2(B)) to determine steady-state concentrations. Subsequently, to achieve a steady state within a target serum level in the range of about 7 mg/ml to about 10 mg/ml, dosing was increased to an 800-mg loading dose and then tapered by 100 mg at each 12-h administration until the 400 mg dosage, which was then administered for the remainder of the 7-day dosing period. Drug or placebo infusions were continued for a total of 14 doses i.e., for 7 days after injury (FIG. 1). All infusions were administered through a subclavian central venous catheter for a 30-min period.

Spinal CSF pressure was transduced through an indwelling lumbar catheter at L4/5, while mean arterial blood pressure was monitored through an indwelling radial artery catheter. Spinal cord perfusion pressure, the difference between mean arterial pressure and CSF pressure, was calculated and monitored electronically in real time. Subjects whose spinal cord perfusion pressure fell below 75 mmHg at any time during the 7 days of treatment underwent a second randomization assigning them to blood pressure maintenance (control) versus spinal cord perfusion pressure augmentation. Those assigned to the control group received crystalloid fluid and inotrope therapy (i.e., norepinephrine) as necessary to maintain mean arterial blood pressure at about 465 mmHg. Those randomized to active spinal cord perfusion pressure augmentation received crystalloid fluid and if necessary, inotrope therapy (i.e., norepinephrine) to maintain spinal cord perfusion pressure at about 475 mmHg. Spinal cord perfusion pressure support was continued until the end of Day 7.

CSF samples (up to 10 ml) were drawn from each subject's indwelling lumbar catheter every 12 h for 7 days as follows: (i) 0.5 h before drug infusion, (ii) 0.5 h after drug infusion, and (iii) 6 h after drug infusion. Each sample was centrifuged at 2000 rpm for 5 min to separate cellular matter. The supernatant was flash frozen and stored at −80° C. in aliquots.

### Clinical Outcome Measures

Neurological function was assessed at intervals using the American Spinal Cord Injury Association (ASIA) standardized neurological examination and included the motor composites and sensory composites. These examinations were performed by a physical medicine and rehabilitation specialist at Day 1 (time of enrolment), Day 4, Day 5, Day 7, Week 3, Week 6, Week 12, Month 6, and Month 12 (Table 3). Day 1 scores were subtracted from each subsequent score to calculate improvement from baseline for graphing purposes.

TABLE 3
Patient follow-up details

| | | | Motor- | Central |
|---|---|---|---|---|
| Time | All subjects | Motor-complete | incomplete | cord injury |
| (days) | (n = 52) | (n = 36) | (n = 10) | (n = 6) |
| 4 | 38* (73%)** | 26 (72%) | 8 (80%) | 4 (67%) |
| 7 | 42 (81%) | 29 (81%) | 8 (80%) | 5 (83%) |
| 21 | 46 (88%) | 32 (89%) | 8 (80%) | 6 (100%) |
| 42 | 39 (75%) | 26 (72%) | 9 (90%) | 4 (67%) |
| 90 | 40 (77%) | 26 (72%) | 9 (90%) | 5 (83%) |
| 182 | 39 (75%) | 27 (75%) | 8 (80%) | 4 (67%) |
| 365 | 40 (77%) | 26 (72%) | 8 (80%) | 6 (100%) |
| Mean % | (78%) | (76%) | (83%) | (81%) |

*number of subjects assessed at each time period.
**percentage of subjects assessed at each time period.

We chose to use the Day 1 score for baseline comparison acknowledging controversy that such early examinations can be prone to more variability. In order to be enrolled in this study, each subject was required to provide an accurate ASIA neurological exam. Thus, 100% of the enrolled subjects had a Day 1 baseline ASIA score. We considered the alternative of using the Day 4 score to adjust for baseline. However, we found that Day 4 examinations were sometimes not possible, in particular, as subjects were in the ICU and ventilated, often with sedation. Consequently, only 73% of subjects had data at Day 4 (FIG. 1, Table 3). Furthermore, it appeared that at Day 4 subject motivation was more likely to influence compliance with the examination. In comparing variability of the Day 1 and Day 4 examination standard deviations (SD) were 22.68 and 24.73, respectively. The mean change in motor score at Day 4 was −0.368 (SD=7.964). Sixty-six percent of subjects displayed a Day 4 motor score within three points of the Day 1 score. Thirteen percent (n=5) displayed a change 410 points.

Functional outcome was assessed using the Spinal Cord Independence Measure, Functional Independence Measure, London Handicap scale, and Short Form 36 questionnaires administered at 6 weeks, 12 weeks, 26 weeks, and 52 weeks after injury.

Subjects were evaluated for adverse events daily while in hospital and at each subsequent clinical evaluation. All serious adverse events (Table 4) were reviewed promptly by a safety monitoring board composed of clinicians and clinician researchers not otherwise involved in this study. A summary of all adverse events was reviewed every 6 months.

TABLE 4
Adverse events by treatment group

| | Placebo* | | | Low dose** | | | High dose*** | | | |
|---|---|---|---|---|---|---|---|---|---|---|
| | Events | # of | # of | Events | # of | # of | Events | # of | # of | |
| System/category | subject | events | subjects | subject | events | subjects | subject | events | subjects | P^{†} |
| Cardiac | 1.10 | 22 | 12 | 1.0 | 5 | 4 | 0.88 | 14 | 9 | 0.787 |
| Respiratory | 0.80 | 16 | 13 | 1.0 | 5 | 4 | 0.81 | 13 | 9 | 0.875 |
| Gastrointestinal | 1.45 | 35 | 15 | 2.2 | 12 | 5 | 1.88 | 36 | 12 | 0.439 |
| Genito-urinary | 0.40 | 8 | 7 | 0 | 0 | 0 | 0.44 | 7 | 5 | 0.326 |
| Musculoskeletal | 0.65 | 14 | 11 | 0.6 | 4 | 4 | 0.75 | 14 | 13 | 0.849 |
| Integumentary | 1.30 | 27 | 14 | 1.0 | 5 | 4 | 1.13 | 21 | 11 | 0.857 |
| Haematological | 0.60 | 12 | 9 | 0.4 | 2 | 2 | 0.63 | 9 | 6 | 0.872 |
| Endocrine | 0.05 | 1 | 1 | 0.2 | 1 | 1 | 0 | 0 | 0 | 0.205 |
| Psychiatric | 0.70 | 15 | 9 | 1.2 | 8 | 4 | 1.13 | 18 | 8 | 0.461 |
| Neurological | 1.45 | 35 | 13 | 1.6 | 11 | 5 | 0.56 | 16 | 10 | 0.073 |
| Infectious | 2.10 | 58 | 19 | 1.8 | 12 | 4 | 1.38 | 28 | 11 | 0.295 |
| Deep venous | 0.10 | 2 | 2 | 0 | 0 | 0 | 0.06 | 1 | 1 | 0.744 |
| thrombosis | | | | | | | | | | |
| Autonomic | 0.10 | 2 | 2 | 0 | 0 | 0 | 0.06 | 1 | 1 | 0.744 |
| dysreflexia | | | | | | | | | | |
| Pain | 2.10 | 44 | 18 | 2.0 | 10 | 5 | 2.00 | 31 | 14 | 0.968 |
| Other | 1.05 | 23 | 14 | 2.4 | 13 | 5 | 0.88 | 19 | 11 | 0.019 |
| Serious adverse | 0.30 | 6 | 5 | 0.2 | 1 | 1 | 0.06 | 1 | 1 | 0.312 |
| events**** | | | | | | | | | | |

*Placebo = normal saline.
**Low dose = 200 mg minocycline administered by IV twice daily for 7 days.
***High dose = 800 mg minocycline initial dose tapered by 100 mg at each 12-h administration until the 400 mg dose. Subsequent doses for the remainder of the 7-day period were 400 mg.
****A serious adverse event was defined as any untoward medical occurrence that results in death or is life threatening or requires inpatient hospitalization or results in prolongation of existing hospitalization ore results in persistent or significant disability and/or incapacity.
^{†}Adverse events/patients in each group were compared by ANOVA.

### Statistical Analyses

Unadjusted ASIA motor scores were compared across time and between groups using repeated measures regression employing data from Day 90, Day 182 and Day 365 with baseline score as a covariate using the ‘R’ statistical package. The model assumed that any change associated with treatment group was constant over these time points. An interaction term between treatment and injury type was included to allow for the possibility that changes associated with treatment group differed among motor-complete and motor-incomplete injured subjects. ASIA sensory scores were similarly evaluated. ASIA motor, pin-prick and light scores were evaluated by the Shapiro-Wilk normality test and were consistent with a normal distribution. Functional recovery data were compared over all data points. Adverse events were categorized by system and mean numbers of events per patient were compared within each system by ANOVA. All statistical tests were two-tailed (a=0.05). Post hoc, we calculated the observed number of subjects and the standard deviation for motor recovery (defined as the average of the motor scores at 3 months, 6 months, and 12 months when plateaus in recovery were seen) for each subgroup analysed. The Student t-test was then used to estimate the between group difference that this study was powered to detect with power=0.8 and a=0.05.

### Results

### Subjects

Fifty-two subjects were entered into the study. An additional 19 subjects (27%) with SCI who presented during the enrolment period were not enrolled; 1.4 subjects (20%) of those did not satisfy the inclusion and exclusion criteria (FIG. 1, Table 1). The study was terminated upon recruitment of 1.0 motor-incomplete subjects, a recruitment target defined prior to the trial as a minimum sample size for outcome evaluation in this key subgroup. Average age was 37 years. Seventy-seven percent of enrolled subjects were male. Thirty-six subjects suffered motor-complete SCI while 10 were motor-incomplete (Table 3). Six patients presented with central cord syndrome (Table 3). Baseline demographic and clinical characteristics for the minocycline and placebo groups are summarized in Table 2. Only subjects presenting within 12 h of injury were included in the study. The mean time from injury to presentation was 3.6 h. The intervention treatments (minocycline or placebo), on average, were started 10.2 h after injury.

Surgical decompression was undertaken within 24 h of injury. The mean time to decompression in our cohort was 17.4 h (Table 2). There were eight (15%) violations to this requirement; four subjects underwent surgery during the 25^{th} hour and two subjects underwent surgery during the 29^{th} hour respectively. These violations occurred due to operating room triaging that resulted in unavoidable delays. Two additional violations were due to surgeon non-compliance with the protocol: (i) one subject with a motor-complete SCI (ASIA A) underwent surgery at 5.5 days, while (ii) the other with central cord syndrome underwent surgery at 11 days. Two subjects did not undergo surgical decompression. One subject with cervical motor-incomplete injury (placebo group) presented with a unilateral facet dislocation that was reduced with traction within 24 h of the injury. Another subject with cervical motor-incomplete injury (minocycline group) was managed in a hard cervical collar as there was no evidence of spinal cord compression or instability on imaging.

None of the subjects enrolled in this study withdrew before completion of their study intervention. There were three protocol violations related to the interventions. A dose error at one time-point occurred when the full daily minocycline dose was administered at once rather than in two divided doses. In two instances, lumbar drain dislodgement occurred requiring replacement. Outcome data were available for 78% of subjects at each time-point (summarized in FIG. 1).

The first five patients to receive minocycline infusions were administered 200 mg twice-daily intravenously, the maximum human dose previously reported. Serum analyses demonstrated a resulting steady-state concentration of 4.2 mg/ml (95% confidence interval (CI) 3.7-4.7) within 48 h (FIG. 2(A)), while CSF samples revealed a steady-state concentration of 1.0 mg/ml (95% CI 0.9-1.1) (FIG. 2(B)). Subsequent minocycline infusions included a loading dose of 800 mg followed by sequential tapering by 100 mg every 12 h until the 400 mg dosage was reached, which was then administered at 12-h intervals until the completion of the Day 7 doses. This dosing schedule achieved a serum steady-state concentration of 12.7 mg/ml (95% CI 11.6-13.8; FIG. 2(A)) and a CSF steady state of 2.3 mg/ml (95% CI 2.1-2.5; FIG. 2(B)) within 24 h.

Two subjects died during the study; one early death (Day 20) in a patient suffering high cervical quadriplegia (placebo group) was attributed to multisystem organ failure and fulminate acute respiratory distress syndrome. Another subject (high-dose minocycline group) died of a narcotic drug overdose at 6 months. Adverse events did not vary significantly among the placebo, low-dose (200 mg) or high-dose (400 mg) minocycline groups (Table 4). Notably, one subject in the high-dose minocycline group displayed elevated liver enzymes, but was otherwise not symptomatic. These indices promptly normalized following discontinuation of the drug. This was the only adverse event likely related to minocycline observed during the study.

### Neurological Recovery

Neurological recovery was followed using the ASIA neurological exam. Motor recovery reached a plateau about 3 months after the occurrence of SCI. This pattern of recovery occurred regardless of whether the injury was motor-complete, motor-incomplete or of the central cord type (FIG. 3). As expected, patients with motor-incomplete injuries recovered more function than those with motor-complete injuries. Those with central cord injuries appeared to display even greater motor recovery. However, the data for that cohort came from only two placebo subjects. Given this limitation, we did not analyse the central cord syndrome group further.

End-point motor recovery for each patient was defined by the plateau in motor function observed in the study population (i.e. 3-12 month outcome data). In the 44 subjects with data available beyond 3 months, this recovery was 6 (95% CI of 3 to 14; P=0.20) motor points greater in patients treated with minocycline than in those receiving placebo (95% CI of 3 to 14; P=0.20) (FIG. 4(A)). Those with thoracic SCI (n=17) did not show any benefit associated with treatment (FIG. 4(B)). However, in the setting of cervical SCI (n=25), minocycline administration was associated with a 14 point difference in motor score over that seen with placebo that approached significance (95% CI 0-28; P=0.05) (FIG. 4(C)). The distribution of motor-complete injury among subjects with cervical injury (versus motor incomplete) differed between the treatment (44%) and placebo (68%) groups possibly affecting this observed difference (Table 3). However, the difference was maintained on subgroup analysis (although not statistically significant) suggesting that the effect of baseline differences in injury severity was not enough to explain this observation. The difference appeared less pronounced in the cervical motor-complete subjects (10 points; 95% CI—9 to 28; P=0.29, n=16) (FIG. 4(D)) than motor-incomplete patients (22 points; 95% CI—7 to 52; P=0.12, n=9) (FIG. 4(E)). However, notable in the cervical motor-incomplete group was one subject administered placebo who experienced exceptionally poor recovery. This subject exhibited a C3 injury level, with an ASIA motor score of 2 initially and 0 on subsequent exams. On exclusion of this subject, an augmented difference between minocycline and placebo in motor-incomplete subjects became less clear (13 points; 95% CI 6 to 31; P=0.135, n=8). Comparison of the low-dose minocycline groups and high-dose minocycline groups suggested a greater difference with higher doses (FIG. 5).

Motor recovery (defined by the plateau in motor function) in the subgroup with cervical SCI was evaluated for the distribution of recovery that differed between the minocycline and placebo groups. We compared motor recovery with and without minocycline in the upper extremities (FIG. 6(A)) and in the lower extremities (FIG. 6(B)). We also compared gain of ASIA motor scores in the zone of partial preservation (myotomes with motor score 55 and 40 at baseline) (FIG. 6(C)) and in new segments in the cervical motor-complete group where these zones could be defined (FIG. 6(D)). These comparisons suggested that the majority of the difference seen between the treatment groups occurred in the lower extremity and in new segment scores. Little difference was seen in the upper extremities or in the zone of partial preservation.

In an attempt to diminish the potential confounding effect of timing to surgical decompression, we undertook surgery within 24 h of injury. Exceptions to this prerequisite are detailed above. We also examined the relationship between timing to surgical decompression and motor recovery using scatter plots and the Person Product-Moment Correlation Coefficient. We did not observe a correlation between the timing of surgical decompression and motor recovery in any subgroup. The correlation coefficients were as follows: (i) all subjects, 0.156, (ii) cervical SCI, 0.025, (iii) thoracic SCI 0.295, (iv) motor-complete SCI, 0.130, (v) motor-incomplete SCI, 0.019, (vi) cervical motor-complete SCI, 0.023, and (vii) cervical motor-incomplete SCI, 0.019.

Sensory recovery followed a time-course and pattern similar to motor recovery. The degree of recovery appeared greater in minocycline-treated patients compared to placebo; however, this was not statistically significant; nine pinprick points (95% CI 3 to 22; P=0.15) (FIG. 8); seven light-touch points (95% CI—6 to 20; P=0.27) (FIG. 7). No difference in sensory scores was apparent between patients with thoracic SCI receiving minocycline or placebo treatments. A tendency towards a higher pinprick and light-touch scores was observed in minocycline-treated patients with cervical motor-incomplete injuries, but this did not reach statistical significance; 14 pinprick points (95% CI—32 to 60, P=0.49) and 20 light-touch points (95% CI—14 to 54, P=0.20).

### Functional Recovery

Functional recovery was assessed using standardized outcome scales: Spinal Cord Independence Measure (FIGS. 9(A)-9(C)), Functional Independence Measure (FIGS. 10(A)-10(C)), London Handicap Scale (FIGS. 11(A)-11(C)), and Short Form 36 (FIGS. 12(A)-12(C)). Similar to ASIA motor and sensory recovery, functional outcomes as assessed by these scales, were greater in the minocycline-treated group but the differences were not statistically significant. These differences were most apparent in the Spinal Cord Independence Measure (FIGS. 9(A)-9(C)) and Functional Independence Measure Outcome scales (FIGS. 10(A)-10(C)). Spinal Cord Independence Measure and Functional Independence Measure are more disease specific and concentrate on performance of particular tasks (e.g. activities of daily living, sphincter management and respiration), while the London Handicap scale and the Short Form 36 further emphasize social functioning (e.g. occupation, social integration and economic self-sufficiency). While a plateau in recovery was generally seen in all scales, it was apparent at 6 months in distinction to the 3-month plateau seen with motor recovery. Similar to neurological recovery using ASIA motor and sensory scores, the difference between the minocycline-treated and placebo-treated groups was more apparent in subjects with cervical motor-incomplete SCI.

In conclusion, the minocycline dosing regimes disclosed here provided with improvements in neurological and functional outcomes in human subjects compared with placebo treatments.

1. A dosing regime for providing a therapy for a neurological trauma in a human subject in need thereof, the dosing regime comprising:
administration of a plurality of minocycline doses for a 7-day period wherein an initial minocycline dose is administered within about 12 h after an occurrence of the neurological trauma and the remainder of the plurality of minocycline doses are administered at 12-h intervals;
wherein a steady state level of minocycline is detectable in the subject's serum after administration of three minocycline doses, said steady state minocycline level in the subject's serum in a range of about 7 μg/ml to about 17.5 μg/ml.

2. The dosing regime of claim 1, wherein a steady state level of minocycline is detectable in the subject's cerebrospinal fluid after administration of three minocycline doses, said steady state minocycline level in the subject's cerebrospinal fluid in a range of about 1.5 μg/ml to about 3.0 μg/ml.

3. The dosing regime of claim 1, wherein the initial minocycline dose comprises about 800 mg of minocycline, a second minocycline dose comprises about 700 mg of minocycline, a third minocycline dose comprises about 600 mg of minocycline, a fourth minocycline dose comprises about 500 mg of minocycline, and a fifth minocycline dose comprises about 400 mg of minocycline.

4. The dosing regime of claim 3, wherein a sixth minocycline dose and/or each of the remaining plurality of minocycline doses comprise about 400 mg of minocycline.

5. The dosing regime of claim 1, wherein the initial minocycline dose is administered within about 36 h after an occurrence of the neurological trauma and the remainder of the plurality of minocycline doses are administered at 12-h intervals.

6. The dosing regime of claim 1, wherein the neurological trauma is a spinal cord injury.

7. The dosing regime of claim 1, wherein the neurological trauma is an acute spinal cord injury.

8. The dosing regime of claim 1, wherein the plurality of minocycline doses are administered by one or more of intravenous injections, intrathecal injections, intraosseous injections, intraperitoneal injections, intramuscular injections, and oral administration.

9. The dosing regime of claim 1, wherein a first group of the plurality of minocycline doses is administered by intravenous injections and the remaining group of the plurality of minocycline doses is administered by one or more of intrathecal injections, intraosseous injections, intraperitoneal injections, intramuscular injections, and oral administration.

10. The dosing regime of claim 9, wherein the first group of the plurality of minocycline doses comprises one to five doses.

11. A method for treating a neurological trauma in a human subject, comprising:
intravenously administering to the subject a plurality of minocycline doses for a 7-day period wherein an initial minocycline dose is administered within about 12 h after an occurrence of the neurological trauma and the remainder of the plurality of minocycline doses are administered at 12-h intervals;
collecting a plurality of blood samples from the subject;
separating a serum sample from each of said plurality of blood samples;
assaying each of the serum samples to determine a level of minocycline in each of the serum samples; and
adjusting one or more of said plurality of minocycline doses to maintain a steady state level of minocycline in the subject's serum in a range of about 7 μg/ml to about 10 μg/ml.

12. The method of claim 11, additionally comprising:
collecting a plurality of cerebrospinal fluid samples from the subject;
assaying each of the cerebrospinal fluid samples to determine a level of minocycline in each of the cerebrospinal fluid samples; and
adjusting one or more of said plurality of minocycline doses to maintain a steady state level of minocycline in the subject's cerebrospinal fluid in a range of about 7 μg/ml to about 10 μg/ml.

13. The method of claim 11, wherein the initial minocycline dose comprises about 800 mg of minocycline, a second minocycline dose comprises about 700 mg of minocycline, a third minocycline dose comprises about 600 mg of minocycline, a fourth minocycline dose comprises about 500 mg of minocycline, and a fifth minocycline dose comprises about 400 mg of minocycline.

14. The method of claim 13, wherein a sixth minocycline dose and/or each of the remaining plurality of minocycline doses comprise about 400 mg of minocycline.

15. The method of claim 11, wherein the initial minocycline dose is administered within about 36 h after an occurrence of the neurological trauma and the remainder of the plurality of minocycline doses are administered at 12-h intervals.

16. The method of claim 11, wherein the neurological trauma is a spinal cord injury.

17. The method of claim 11, wherein the neurological trauma is a spinal cord injury.
